(19) United States
(12) Patent Application Publication
(10) Pub. No.: US 2009/0188791 A1
(43) Pub. Date: Jul. 30, 2009

(54) Biosensor and its production

(75) Inventors: Ingo Neubert, Norderstedt (DE); German Patino, Hamburg (DE); Maren Klose, Seevetal (DE); Ilse Rodewald, Hamburg (DE)

Correspondence Address: NORRIS, MCLAUGHLIN & MARCUS, 875 THIRD AVE, 18TH FLOOR, NEW YORK, NY, 10022, US

(73) Assignee: TESA AG, Hamburg (DE)

(21) Appl. No.: 12/052,482

(22) Filed: Mar. 20, 2008

(30) Foreign Application Priority Data
Jan. 25, 2008 (DE) 10 2008 006 225.1

(51) Int. Cl. G01N 33/50

(52) U.S. Cl. 204/403.01

(57) ABSTRACT

Biosensor by means of which biological fluids are analysed, comprising at least the
following layers:
   a functional layer A1 which constitutes the base layer of the biosensor,
   a functional layer A3 which is composed of a polymer film which has at least partially a hydrophilic coating,
   a double-sided pressure-sensitive adhesive tape A2 which joins the functional layers A1 and A3 to one another and in which a measuring channel is provided whose lid is formed by the functional layer A3 and whose base is formed by the functional layer A1,
characterized in that
the polymer film of the functional layer A3 has on its inside, which forms a wall of the measuring channel formed by the functional layer A2, at least one venting notch which is disposed above the measuring channel in such a way that the venting notch vents the measuring channel.

### BACKGROUND OF THE INVENTION

The present invention relates to a biosensor, and to its production, by means of which biological fluids such as blood, urine, saliva or cell fluid, for example, are analysed. In the text below, a biosensor, more generally called microfluidic devices, also embraces analytical test strips, which are also known as medical diagnostic strips.

In modern medical diagnostics an ever greater number of biosensors is being used. With the aid of these biosensors it is possible to analyses for example, biological fluids such as blood, urine and saliva on the one hand for pathogens, incompatibilities, DNA activity or enzyme activity and on the other hand for levels of glucose, cholesterol, proteins, ketones, phenylalanine or enzymes.

On the biosensors, detection reactions or reaction cascades take place. For this purpose the biological test fluid must be transported to the reaction site or to the various reaction sites. The modern biosensors therefore consist of at least one microchannel or microchannel system through which the test fluid is transported. The microchannels typically have a height and width of 5 to 1500 μm. Transport within the channels is accomplished by means of capillary forces or centrifugal forces. The results of the detection reactions are usually read optically or electrochemically.

One of the first patents in the technical field of test strips appeared back in 1964. U.S. Pat. No. 1,073,596 A describes a diagnostic test and the test strips for analysing biological body fluids, especially for determining blood sugar. The diagnostic test functions via the determination of a colour change which is triggered by an enzyme reaction.

Determining a change in concentration of a dye (colorimetric method) is still a method used today in the determination of blood sugar by means of diagnostic test strips. It involves reaction of the enzyme glucose oxidase/peroxidase with the blood sugar. The hydrogen peroxide formed then reacts with an indicator, such as O-tolidines, for example, which leads to a colour reaction. This colour change can be monitored by colorimetric methods. The degree of change in colour is directly proportional to the blood sugar concentration. The enzyme here is located on a woven fabric. This method is described for example in EP 0 451 981 A1 and WO 93/03673 A1.

The modern development of diagnostic test strips aims to shorten the measuring time between the application of blood to the test strip and the appearance of the result. The measuring time, or the time between the application of the blood to the diagnostic measuring strip and the display of the result, is dependent not only on the actual reaction time in the enzyme reaction and in the follow-on reactions, but likewise, to a considerable extent, on the time taken for the blood to be transported within the diagnostic strip from the blood application point to the reaction site, in other words to the enzyme.

One of the ways in which the measuring time is shortened is by the use of hydrophilicized woven or nonwoven fabrics, as in U.S. Pat. No. 6,555,061 B, to transport the blood more quickly to the measuring region (enzyme). The further measuring method is identical with that described in EP 0 451 981 A1. For the construction of the diagnostic strip a double-sided standard adhesive tape (Scotch® 415), is used. Surface-modified woven fabrics having a wicking effect for the biological fluid are described in WO 93/03673 A1, WO 03/067252 A1 and US 2002/0102739 A1. In the latter application, plasma treatment of the woven fabric produces a blood transport rate of 1.0 mm/s. However, when woven fabrics are used for transporting biological test fluid such as blood, for example, a chromatography effect is observed: that is, the individual constituents such as cells are separated from the liquid constituents. The chromatography effect is exploited explicitly in WO 03/008933 A2 for the separate analysis of the blood constituents.

An onward development from the colorimetric measuring method is the electrical determination of the change in oxidation potential at an electrode coated with the enzyme. This method and a corresponding diagnostic test strip are described in WO 01/67099 A1. The diagnostic test strip is constructed by the printing of different functional layers such as interconnects, enzyme and hotmelt adhesive onto the base material made, for example, of polyester. Subsequently an otherwise unspecified hydrophilic film is laminated on by thermal activation of the adhesive. The purpose of the hydrophilic film, here again, is to accelerate the transport of the blood to the measuring cell. With this construction there is no need for woven or nonwoven fabric to transport the blood. The advantage of this construction and the advantage of the new measuring method is that the blood sugar level can be measured with a very much smaller volume of blood, around 5 to 10 μl, and in a shorter measuring time.

U.S. Pat. No. 5,997,817 A describes an electrochemical biosensor in which the transport of the biological fluid is realized likewise by way of a hydrophilic coating. The coating in question is ARCARE 8586 (not available commercially) from Adhesive Research Inc. The transport of the biological fluid is evaluated in a specific capillary test of which, however, no further details are given.

DE 102 34 564 A1 describes a biosensor which is composed of a planar sensor or test strip and a compartmentalized reaction-and-measurement chamber attachment which is produced by embossing of a PVC film. The measurement chamber attachment is composed of a very specific embossed design comprising sample application duct, measurement chamber, sample arrest duct and sample collection space. The embossed depth of this compartmentalized system amounts to 10 to 300 μm. The sample application duct and the measurement chamber are furnished with a woven hydrophilic fabric or with a surfactant coating for the transport of the biological fluid.

DE 10211 204 A1 describes a flow-through measuring cell for the continuous determination of glucose. The measuring cell is composed of a planarly structured film which forms a small inlet duct and a substantially larger outlet duct, the two ducts opening into one another via a defined angle.

Further typical applications include, for example, biosensors (for example U.S. Pat. No. 5,759,364 A1) and blood sugar test strips (for example WO 2005/033698 A1, U.S. Pat. No. 5,997,817 A1).

The majority of these biosensors are composed of a measuring channel into which the biological fluid, the analyte, must flow in order that it can be analyzed for certain substances it may contain, in a detection reaction. Fluid transport is normally ensured by way of a hydrophilic coating. The hydrophilic coating often contains surface-active substances There are various patents which concern themselves with the subject of a hydrophilic coating. Examples include US 2005/0084681 A1, EP 1 647 568 A1, U.S. Pat. No. 6,969,166 B2, US 2002/0110486 A1 and EP 1 394 535 A1.

Within the measuring channel, uncontrolled transport or uncontrolled movement of the biological fluid is undesirable, since this adversely affects the result. Furthermore, the trend is in the direction of the development of biosensors with ever smaller fluid volumes, in order thereby to reduce the pain associated with the taking of blood in the case, for example, of diabetes patients. This object is often achieved by biosensor designs having a measuring channel which is closed at the end, so that the volume of fluid is precisely defined by the geometry of the measuring channel. This, however, produces a further problem. At its closed end, the measuring channel must have a venting means, since otherwise the fluid is unable to flow into the channel.

The problem of the venting of the measuring channel has already been solved and is described in publications including those which now follow. In WO 2004/113901 A1, DE 197 53 851 A1 and WO 99/29429 A1, various cover films are applied over the measuring channel in such a way that, by overlapping or end-to-end bonding, they form a small gap which then serves for the venting of the measuring channel. U.S. Pat. No. 7,086,277 B2, EP 1 156 325 A1, EP 0359 831 A1, U.S. Pat. No. 5,997,817 B2 provide the cover film with an air hole. U.S. Pat. No. 6,939,450 B2 solves the venting problem by embossing in the cover film an air duct which serves as a venting duct.

### SUMMARY OF THE INVENTION

It is an object of the present invention to provide a biosensor which in accordance with the requirements is suitable for the analysis of biological fluids and which specifically ensures rapid transport of the biological fluid into the measuring channel by means of special venting. It must also be ensured here that the properties, and especially the transport properties in the measuring channel of the biosensor, are retained even after a long storage time.

This object is achieved by means of a biosensor as recorded in the main claim. Subject matter of the dependent claims are advantageous developments of the subject matter of the invention. The invention further embraces the possibility for use of the biosensor of the invention inter alia in medical sensors or diagnostic strips for analysing biological fluids.

The invention accordingly provides a biosensor by means of which biological fluids are analysed, comprising at least the following layers:
a functional layer A1 which constitutes the base layer of the biosensor,
a functional layer A3 which is composed of a polymer film which has at least partially a hydrophilic coating, and
a double-sided pressure-sensitive adhesive tape A2 which joins the functional layers A1 and A3 to one another and in which a measuring channel is provided whose lid is formed by the functional layer A3 and whose base is formed by the functional layer A1.
   The polymer film of the functional layer A3 has on the inside, which forms a wall of the measuring channel formed by the functional layer A2, at least one, preferably precisely one, venting notch which is arranged above the measuring channel in such a way that the venting notch vents the measuring channel. This venting notch allows the otherwise closed measuring channel to be filled with the biological fluid, since the air displaced by the fluid is able to escape from the measuring channel through the venting notch.

In one particularly advantageous embodiment of the invention the measuring channel, which is formed by the functional layers A1 and A3 and also by the pressure-sensitive adhesive tape A2, which is present in the form of a diecut, has only at the front side of the biosensor an orifice which constitutes the application orifice for the biological fluid. At the other end, in the interior of the biosensor, the measuring channel is closed. Furthermore, the measuring channel is vented only by the venting notch on at least one side of the biosensor, the venting notch in the polymer film of the functional layer A3 being positioned above the measuring channel in such a way that it is located approximately at the end of the measuring channel.

The venting notch is produced preferably with a slitting or punching tool in the polymer film of the functional layer A3.

The base material used for the functional layer A3 comprises the carrier materials that are typical and familiar to the skilled person, such as films of polyethylene (HDPE, LDPE, MDPE or copolymers of ethylene and of further olefins such as propene, butene, hexene or octene, such as LLDPE or VLLDPE, for example), polypropylene homopolymers, random polypropylene copolymers or block polypropylene copolymers), polyvinyl chloride, polyester; polyacrylate, polycarbonate and, with particular preference, polyethylene terephthalate (PET). The films in question may be monofilms, coextruded or laminated films, which may be unoriented or monoaxially or biaxially oriented. This list is to be understood as being exemplary and not conclusive.

The surface of the films may be microstructured by means of suitable methods such as embossing, etching or laser treatment, for example.

The use of laminates, coextrudates, nonwovens, woven fabrics or membranes is likewise possible.

For better anchorage of the coating, the carrier materials may be chemically or physically pretreated by the standard methods, as for example by corona treatment or flame treatment. To promote adhesion it is likewise possible to prime the carrier material using, for example, PVC, PVDC, polyurethanes or thermoplastic polyester copolymers.

The thickness of the carrier film of the functional layer A3 in one advantageous embodiment of the invention is 12 to 350 μm and more preferably 25 to 150 μm.

The functional layer A3 is preferably provided with a hydrophilic coating or imprint which has a surface tension of at least 60 mN/m and a contact angle with water of less than 30°.

In one further preferred embodiment the thickness of the hydrophilic coating is not more than 3 μm and advantageously not more than 1.5 μm.

The functional layer A3 has preferably a hydrophilic coating, applied at least partially, and a venting notch. The hydrophilic coating ensures on the one hand that the test fluid flows into the measuring channel. For this purpose the hydrophilic region ought to extend up to the front edge of the entry orifice of the measuring channel. If this is not the case it is impossible for the test fluid to be transported into the measuring channel, particularly if it is a fluid having a relatively high viscosity such as blood, for example. On the other hand, the hydrophilic region is likewise important for the very rapid transport of the test fluid into the measuring channel, so that measurement can be commenced as quickly as possible, in order thus to be able to realize an extremely short measuring time. The measuring times in blood sugar test strips on the market at present amount to three seconds.

The hydrophilic region is applied either over the full area or partially to the functional layer A3. Full-area application takes place advantageously in a coating process. Examples of suitable coating processes include spray coating, halftone roller application, Mayer bar coating, multi-roll application coating, condensation coating or else printing processes. Partial coating takes place preferably with a printing process, preferably in flexographic printing. In this case the viscosity of the coating solution is adapted to suit the printing process. This is typically done using a polymer as binder.

The coating is preferably composed of at least one surfactant, which is preferably an anionic surfactant and more preferably a surfactant based on a sulphosuccinic ester salt.

The surfactant is critically responsible for the hydrophilic properties. Surfactants which can be used include compounds comprising linear or branched alkyl, alkylbenzyl, perfluorinated alkyl or siloxane groups with hydrophilic head groups, such as anionic salts of carboxylic acids, phosphoric acids, phosphonic acids, sulphates, sulphonic acids, sulphosuccinic acid, cationic ammonium salts or nonionic polyglycosides, polyamines, polyglycol esters, polyglycol ethers, polyglycol amines, polyfunctional alcohols or alcohol ethoxylates. This selection is an exemplary enumeration and does not represent any restriction of the inventive concept to the surfactants specified. By way of example the following suitable surfactants may be specified:
nonionic fatty alcohol ethoxylated surfactants, for example Tego Surten® W111 from
Evonik AG or Triton® X-100 and Tergitol® 15-S from Dow Chemicals Inc.
nonionic fluoro surfactants, for example Fluorad® FC-4430 and FC-4432 from 3M Inc., Zonyl® FSO-100 from DuPont Inc. and Licowet® F 40 from Clariant AG nonionic silicone surfactants, for example Q2-5211 and Sylgard® 309 from Dow Corning Inc., Lambent® 703 from Lambent Technologie Inc. and Tegopren® 5840 from Evonik AG
ionic alkyl sulphate salt, for example Rewopol® NLS 28 from Evonik GmbH
ionic sulphosuccinic salts, for example Lutensit® A-BO from BASF AG or Rewopol® SB DO from Evonik GmbH

Particular preference is given to using an ionic sulphosuccinic salt and with very particular preference sodium diisooctylsulphosuccinate (CAS number 577-11-7) as surfactant. The ionic sulphosuccinic salts are particularly suitable on account of their very good wetting behaviour with very good ageing resistance and low mobility. The very good wetting behaviour is exhibited in a surface tension of at least 60 mN/m and in a contact angle with water of less than 30°. Nor is there any change in the wetting behaviour after a long storage time, which can be simulated by accelerated ageing at elevated temperatures, for example 70° C. Low mobility is necessary in the surfactant in order to avoid transfer of the surfactant to guide rolls in the production and processing operation.

The coating for the hydrophilic region may advantageously likewise comprise at least one polymer as a binder. As the polymer it is possible to use all of the film-forming binders that are known from the printing inks industry. Advantageously the binder used will be a polymer having polar functional groups such as, for example, hydroxyl, carboxyl, ether, ester, amine, amide groups. Suitable binders that may be mentioned, by way of example and without restriction, are homopolymers or copolymers such as polyvinylpyrrolidone, polyvinylbutyral, polyesters, polyacrylate, polyacrylic acid, polyvinyl acetate, polyvinyl alcohol, polyacrylamide, polyamide, polyethylene glycol, polypropylene glycol, cellulose derivatives.

Advantageously water is used as a solvent for the hydrophilic coating. The binder ought therefore to be water-soluble.

In one preferred variant of the coating varnish of the invention a polyvinyl alcohol is used as binder.

The hydrophilic coating may likewise comprise further additives such as organic dyes or inorganic pigments, ageing inhibitors and/or fillers (in this regard see “Plastics Additives Handbook”, sections “Antioxidants”, “Colorants”, “Fillers”, Carl Hanser Verlag, 5th edition).

The aqueous coating solution is applied in the form of a full-area or partial coating in a printing process; preferably in flexographic printing.

The functional layer A3 is provided at least with one uninterrupted venting notch, which is produced advantageously using a rotary punching tool with a continuous punching blade. The venting notch is located as an indentation in the polymer film of the functional layer A3. The venting notch in the polymer film is positioned above the measuring channel in such a way that it is advantageously located approximately at the opposite end from the application orifice of the measuring channel, and points to the inside of the measuring channel.

When selecting the depth of the venting notch it should be borne in mind that, as the depth increases, the strength of the carrier material is reduced. If the notching of the functional layer A3 is too deep, untroubled processing of the material is no longer possible. On the other hand, an inadequate depth of the venting duct leads to impairment or to the loss of the functionality of the biosensor, since in that case the measuring channel is no longer adequately vented and, as a result, the test fluid is no longer able to flow at all, or is able to flow only very slowly, into the measuring channel. The loss of functionality occurs in particular after storage.

The venting notch in the functional layer A3 therefore preferably has a depth of 10 μm up to a maximum of 90% of the thickness of the polymer film, more preferably of 40 μm up to a maximum of 75% of the thickness of the polymer film.

The depth of the venting duct is determined via the depth of penetration of the cutting tool. The height profile of the punching tool is set and monitored accordingly by the tool manufacturer. Corresponding cutting and punching tools can be obtained, for example, from Rotometrix GmbH, Spilker GmbH, Schober GmbH and Electo Optic GmbH.

The notching operation, in other words the production of the venting notch in the functional layer A3, may take place at different points in the production operation. The carrier may be notched before or after hydrophilic coating or directly in the laminating step with the functional layer A2. For this purpose it is possible to use all commercially customary cutting, notching or punching processes which are accompanied by the required precision for the notching operation. Suitability for this purpose is possessed preferably by the method of rotary punching, which is carried out inline in the laminating step with the functional layer A2.

Preferably the notch extends, starting from the end of the measuring channel in the biosensor, in a straight line, in the transverse or longitudinal direction of the biosensor, up to the outside edge of the biosensor.

The notch may alternatively extend over the entire length or width of the functional layer A3 and hence of the biosensor; what is essential is that the notch intersects the measuring channel in the vicinity of the closed end.

The present invention is not confined to a linear venting notch. Instead, the venting notch may likewise be made, using a suitable tool, in the form of a serpentine line or a zigzag line. The cross section of the notch advantageously tapers to a point from the outside of the polymer film towards the inside of the biosensor. The width of the venting notch at the broadest point (of the polymer film surface) is advantageously not more than 250 μm and with particular advantage not more than 150 μm.

The notch preferably has a triangular profile, the width of the base of the profile corresponding approximately to the depth of the profile.

With further advantage the pressure-sensitive adhesive tape A2 is composed of a pressure-sensitive adhesive having a high shear strength, its holding power at 25° C., 40° C. and 70° C. under a weight load of 1000 g being greater than 10 000 min, and its shear deformation after 15 min at 40° C. under a load of 500 g being less than 130 μm and preferably less than 80 μm.

Provided in the pressure-sensitive adhesive tape is the measuring channel, its two other walls formed by the functional layers A1 and A3. Advantageously the measuring channel is punched out in a diecutting process from the pressure-sensitive adhesive tape strip or diecut.

With particular advantage the pressure-sensitive adhesive tape forms a measuring channel comprising two parallel side walls, the measuring channel being open only at one side.

The pressure-sensitive adhesive tape of the functional layer A2 may be composed not only of one or more layers of a pressure-sensitive adhesive transfer tape (pressure-sensitive adhesive tape without carrier film), which may be laminated with carrier films, but also of a double-sided pressure-sensitive adhesive tape consisting of a carrier film coated on both sides with the pressure-sensitive adhesive.

The adhesives and the application rates of adhesive on the top and bottom sides of the pressure-sensitive adhesive tape may be identical, but can also be selected differently, in order to meet the particular requirements.

The sum of the application rates of the layers of adhesive in the functional layer A2 is in one advantageous embodiment not more than 70 g/m² and preferably not more than 50 g/m².

With further preference the pressure-sensitive adhesive tape is composed of a polyester carrier film coated on each side advantageously with not more than 35 g/m² and more preferably with not more than 25 g/m² of a pressure-sensitive adhesive.

The characteristic quality of the biosensor of the invention is a function of the combination between the pressure-sensitive adhesive tape of the functional layer A2 and the functional layer A3, the pressure-sensitive adhesive or pressure-sensitive adhesive of the pressure-sensitive adhesive tape exhibiting a high level of cohesion or shear strength, respectively. The use of a pressure-sensitive adhesive with a high shear strength is necessary in order to avoid residues of adhesive in the biosensor production process and so to make the production process efficient, and also to avoid penetration of the pressure-sensitive adhesive into the venting notch. Generally speaking, pressure-sensitive adhesives always have a certain flow behaviour, referred to as cold flow. In the case of standard adhesives with a moderate shear strength, this cold flow is considerably more pronounced than in the case of high-shear-strength pressure-sensitive adhesives. As a result of the cold flow, punched shapes do not retain their dimensional accuracy with low tolerances over the storage time, as would be important for the production of a highly precise measuring channel, and, furthermore, unevenesses are compensated over time with the pressure-sensitive adhesive. Owing to the last point, the use of a standard adhesive may be accompanied by clogging or plugging of the venting notch as a result of the cold flow. Surprisingly, in the case of the biosensor of the invention, with the preferred, high-shear-strength pressure-sensitive adhesive, there is no observed clogging of the venting duct, not even after a storage time of 6 weeks at elevated storage temperatures of 40 and 70° C. The high shear strength of the pressure-sensitive adhesive is expressed in a high holding power of more than 10 000 min at 70° C. under a weight load of 1000 g and also in a shear deformation after 15 min at 40° C. under a load of 500 g of less than 130 μm and preferably less than 80 μm. Additionally, the pressure-sensitive adhesive must have sufficient bond strength to prevent delamination of the functional layers and also to prevent the test fluid running down between the pressure-sensitive adhesive tape and the functional layer.

Suitability for preparing the adhesive of the pressure-sensitive adhesive tape with the properties described is possessed by copolymers or copolymer blends of acrylate monomers, or styrene block copolymers with, for example, ethylene, propylene, butylene, butadiene, hexene and/or hexadiene as comonomers.

In the preferred version the pressure-sensitive adhesive of the pressure-sensitive adhesive tape is composed of one or more copolymers of at least the following monomers:
c1) 70% to 100% by weight of acrylic esters and/or methacrylic esters and/or their free acids, with the following formula:

CH₂═CH(R₁)(COOR₂),
where R₁ is H and/or OH₃ and R₂ is H and/or alkyl chains having 1 to 30 C atoms.

Here as well it is possible for the parent monomer mixture to have had c2) up to 30% by weight of olefinically unsaturated monomers with functional groups added to it as a further component.

In one very preferred embodiment use is made for the monomers c1) of acrylic monomers which comprise acrylic and methacrylic esters with alkyl groups consisting of 4 to 14 C atoms, preferably 4 to 9 C atoms. Specific examples, without wishing to be restricted by this enumeration, are n-butyl acrylate, n-pentyl acrylate, n-hexyl acrylate, n-heptyl acrylate, n-octyl acrylate, n-nonyl acrylate, lauryl acrylate, stearyl acrylate, behenyl acrylate, and their branched isomers such as, for example, t-butyl acrylate and 2-ethylhexyl acrylate.

Further classes of compound which may likewise be added in small amounts under c1) are methyl methacrylates, cyclohexyl methacrylates, isobornyl acrylate and isobornyl methacrylates.

In one very preferred embodiment use is made for the monomers c2) of vinyl esters, vinyl ethers, vinyl halides, vinylidene halides, vinyl compounds with aromatic rings and heterocycles in α position.

Here again mention may be made of a number of examples, without the enumeration being considered conclusive:

vinyl acetate, vinylformamide, vinylpyridine, ethyl vinyl ether, vinyl chloride, vinylidene chloride and acrylonitrile.

In a further very preferred embodiment use is made for the monomers c2) of monomers having the following functional groups:

hydroxyl, carboxyl, epoxy, acid amide, isocyanato or amino groups.

In one advantageous variant there are for c2) acrylic monomers Corresponding to the general formula

CH₂═CH(R₁)(COOR₃),
where R₁ is H or CH₃ and the radical R₃ represents or constitutes a functional group which supports subsequent UV crosslinking of the pressure-sensitive adhesive—which for example, in one particularly preferred embodiment, possesses an H donor effect.

Particularly preferred examples for component c2) are hydroxyethyl acrylate, hydroxypropyl acrylate, hydroxyethyl methacrylate, hydroxypropyl methacrylate, allyl alcohol, maleic anhydride, itaconic anhydride, itaconic acid, acrylamide and glyceridyl methacrylate, benzyl acrylate, benzyl methacrylate, phenyl acrylate, phenyl methacrylate, t-butylphenyl acrylate, t-butylphenyl methacrylate, phenoxyethyl acrylate, phenoxyethyl methacrylate, 2-butoxyethyl methacrylate, 2-butoxyethyl acrylate, dimethylaminoethyl methacrylate, dimethylaminoethyl acrylate, diethylaminoethyl methacrylate, diethylaminoethyl acrylate, cyanoethyl methacrylate, cyanoethyl acrylate, gyceryl methacrylate, 6-hydroxyhexyl methacrylate, N-tert-butylacrylamide, N-methylolmethacrylamide, N-(butoxymethyl)methacrylamide, N-methylolacrylamide, N-(ethoxymethyl)-acrylamide, N-isopropylacrylamide, vinylacetic acid, tetrahydrofurfuryl acrylate, β-acryloyloxypropionic acid, trichloroacrylic acid, fumaric acid, crotonic acid, aconitic acid, dimethylacrylic acid, where this enumeration should not be understood as being conclusive.

In a further preferred embodiment use is made for component c2) of aromatic vinyl compounds, where the aromatic nuclei are preferably composed of C₄ to C₁₈ and may also include heteroatoms. Particularly preferred examples are styrene, 4-vinylpyridine, N-vinylphthalimide, methylstyrene, 3,4-dimethoxystyrene, 4-vinylbenzoic acid, where this enumeration should not be considered as being conclusive.

For preparing the polyacrylate pressure-sensitive adhesives it is advantageous to carry out conventional free-radical polymerizations or controlled free-radical polymerizations. For the polymerizations which proceed by a radical mechanism it is preferred to use initiator systems which in addition contain further free-radical initiators for the polymerization, more particularly thermally decomposing free-radical-forming azo or peroxo initiators. In principle, however, all typical initiators familiar to the skilled person for acrylates are suitable. The production of C-centred free radicals is described in Houben Weyl, Methoden der Organischen Chemie, Vol. E 19a, pages 60 to 147. These methods are preferentially employed in analogy.

For the advantageous ongoing development no additives at all such as tackifying resins or plasticizers are added to the polyacrylate pressure-sensitive adhesive of the pressure-sensitive adhesive tape. Although such additives increase the bond strength they may considerably reduce the shear strength of the pressure-sensitive adhesive and so may lead to residues of adhesive on the slitting tools during the operation of slitting the biosensors.

In summary the preferred embodiment of the pressure-sensitive adhesive tape features a polyacrylate pressure-sensitive adhesive which is manufactured by coextrusion or by coating from the melt, solvent or dispersion. Particular preference is given to comma bar coating of the polyacrylate pressure-sensitive adhesive from a suitable solvent or solvent mixture.

The pressure-sensitive adhesive tape of the invention may optionally comprise a carrier film coated on both sides with the pressure-sensitive adhesive. Carrier materials used are the typical carrier materials familiar to the skilled person, such as films of polyester, polyethylene, polypropylene, oriented polypropylene, polyvinyl chloride, more preferably films of polyethylene terephthalate (PET). This enumeration should not be considered as being conclusive; instead, in the context of the invention, other films are included. In order to enhance the adhesion of the adhesive to the carrier film and hence to avoid residues of adhesive on the slitting tool during the operation of slitting the biosensors it is possible to use a primer layer between carrier film and pressure-sensitive adhesive or, preferably, to perform a physical surface treatment such as flaming, corona or plasma on the carrier film.

Advantageously, diecuts having a diecut shape suitable for the application are produced from the pressure-sensitive adhesive tape. The diecut from the pressure-sensitive adhesive tape in this case forms the measuring channel and, accordingly, the functional layer A2. The diecut shape preferably forms a cutout comprising two parallel walls, which is open towards one end of the orifice for application of the test fluid (see FIG. 1a in this regard). Other diecut designs are possible within the bounds of this invention. The pressure-sensitive adhesive tape diecuts are produced using the customary methods such as flat-bed diecutting, rotary diecutting, ultrasonic slitting, water-jet slitting or laser slitting. Production of the diecuts requires a very high level of precision, in the μm range. The diecut produced from the pressure-sensitive adhesive tape can be laminated, immediately after the diecutting operation, to the functional layer A3, thereby allowing the combination of the diecut of the pressure-sensitive adhesive tape (functional layer A2) and the functional layer A3 to be supplied directly to the biosensor production process. It is also possible, however, for the pressure-sensitive adhesive tape and the functional layer A3 to be supplied separately to the biosensor production process and to be laminated to one another only once they are there. The diecuts are preferably produced in the form of continuous rolls, in a so-called roll-to-roll operation, without being separated. In this case only the future measuring channel is cut out. With particular preference, in the same operation of the diecutting process, as described, the functional layer A3 is laminated onto the functional layer A2. In a corresponding inline operation, in other words in the same operating step, the hydrophilic coating and the venting notch of the functional layer A3 can be positioned optimally onto the diecuts of the functional layer A2. Precise positioning is vital for the functioning of the biosensor of the invention. It is particularly advantageous if the hydrophilic coating and the venting notch of the functional layer A3 are likewise produced inline in the diecutting operation, and the lamination of the functional layers A2 and A3 is likewise carried out inline in this operation. An alternative possibility is for the entire production process of the biosensors, of the production of the interconnects and printing with the enzyme layer (functional layer A1), the production of the diecuts of the functional layer A2, of the hydrophilic coating and of the venting notch of the functional layer A3, and also the lamination of these three functional layers, to take place in one and the same operation. The separation or singularization of the biosensors takes place typically in a separate slitting operation.

With further preference the functional layer A1 is provided with interconnects and at least partially with an analytical detection reagent.

The biosensor of the invention for investigating analytes in biological fluids operates preferably in accordance with an electrochemical (amperometric) measuring method. Preference is given in this context to the detection of glucose in human blood, or blood sugar determination.

The base film, the functional layer A1, consisting for example of PVC, paper, polycarbonate or, preferably, polyester, with a thickness range of 200 to 500 μm, is provided with electrical interconnects. An electrochemical biosensor typically requires working electrode, counterelectrode and, if appropriate, reference electrode. The electrical interconnects can be applied in a printing process such as screen printing, for example, to the base film. This is done using conductive pastes, which for example may be conductive carbon, graphite, silver or silver chloride pastes. Depending on the construction there may be insulating layers, likewise applied by printing, between the various interconnect layers. Alternatively the base film can also be laminated, vapour-coated or sputter-coated with a conductive layer of copper, silver, gold or aluminium, for example. In this case the interconnects are obtained in a downstream operation by etching or by a laser ablation process (in this regard see WO2006/074927 A1). Applied to the working electrode and counterelectrode is the enzyme or enzyme mixture needed for the detection reaction and comprising, for example, glucose oxidase or glucose dehydroxygenase and a redox mediator such as, for example, phenanthroline, quinones, ferrocene or derivatives. The detection reagent may further comprise additional additives such as film formers (for example polyvinyl alcohol), enzyme stabilizers (for example glutamate or trehalose), polysaccharides, cellulose derivatives and/or gelatin derivatives. The citable prior art includes WO 2005/033698 A1, WO 2005/101994 A1, U.S. Pat. No. 6,541,216 B1 and EP 1 253 204 A1.

When the biosensor of the invention is used, the filling of the measuring channel with the biological test fluid is very quick. Surprisingly for the skilled person, a simple notch in the cover film ensures the venting of the otherwise closed measuring channel and allows very rapid filling of the measuring channel. A further surprise is that this functionality is fully retained even after a storage time of 6 weeks at elevated temperatures of 70° C. The skilled person would have expected a loss of functionality after such storage, as a result of the clogging of the venting notch.

The inventive biosensor is illustrated below in one particularly outstanding embodiment by means of figures, without wishing the choice of the figures depicted to impose any unnecessary restriction.

### BRIEF DESCRIPTION OF THE DRAWING

In the drawings, wherein like reference numerals delineate similar elements throughout the several views:

FIGS. 1a, 1b, 1c and 1d show two biosensors of particularly advantegous design and

FIG. 2 shows one exemplary production of a venting notch.

### DETAILED DESCRIPTION OF THE PRESENTLY PREFERRED EMBODIMENTS

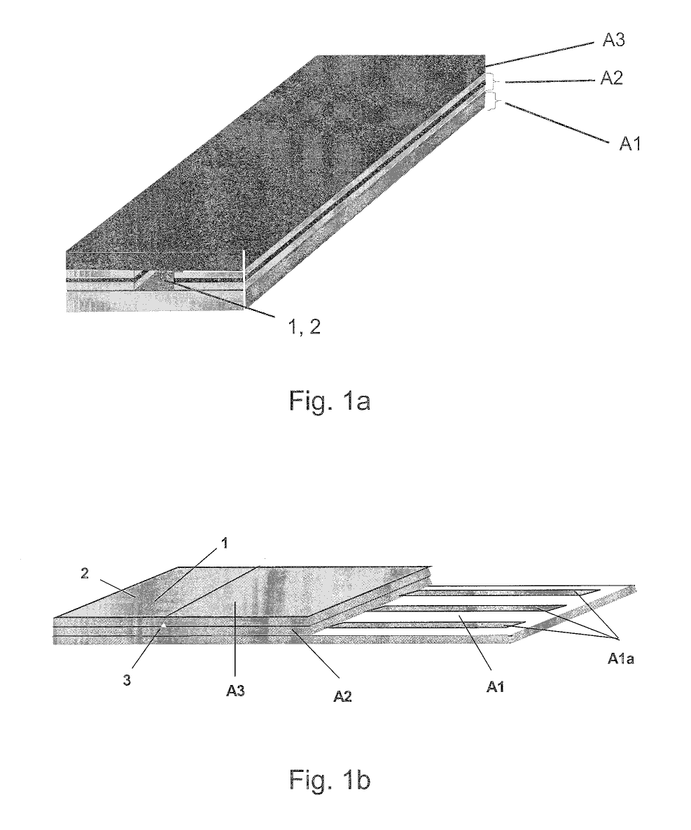

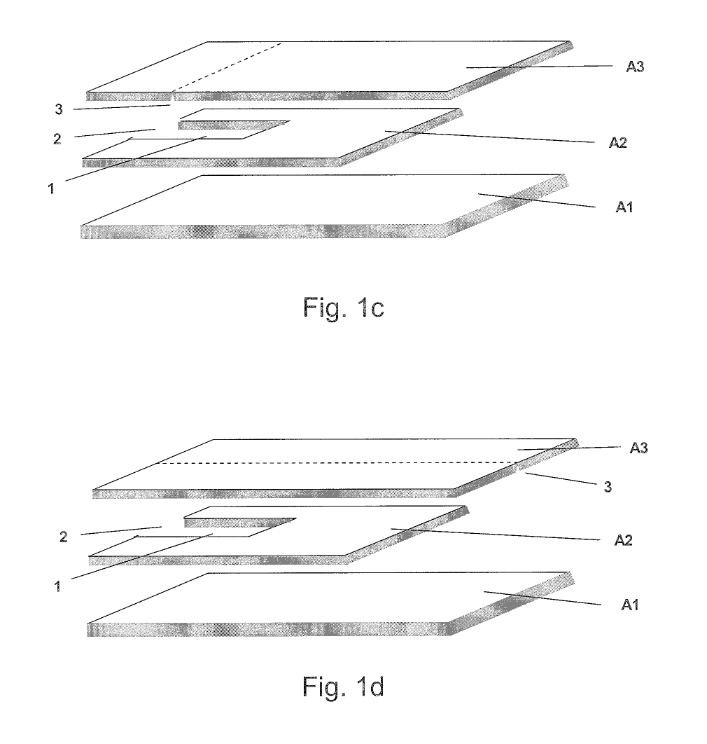

FIGS. 1a, 1b and 1c show by way of example the construction of a biosensor having a measuring channel 1 which is formed by a diecut of a double-sided pressure-sensitive adhesive tape A2. In the front edge of the biosensor the measuring channel 1 has an entry orifice 2, from which the sample fluid is fed into the measuring channel 1. On the side opposite the orifice 2, the measuring channel 1 is closed.

The functional layer A2, or the pressure-sensitive adhesive tape A2, with the measuring channel 1 is laminated to the functional layers A1 and A3. The functional layers A1 and A3 therefore likewise each form a wall of the measuring channel 1. Located on the functional layer A1 are interconnects A1a and also a partial coating of the detection reagent or of the enzyme (not shown in the drawing).

In dimensional terms, the functional layer A1 is longer than the functional layers A2 and A3, so that it protrudes beyond them at least on one side. As a result, the interconnects A1a applied there are accessible, and electrical contact with the read device can be established. The measuring channel 1 is bordered from the opposite side with the functional layer A3 as a further wall. The functional layer A3 is furnished with a hydrophilic coating and also with the venting notch 3. The venting notch 3 is positioned above the measuring channel in such a way that it is located in the vicinity of the end of the measuring channel 1 opposite the entry orifice 2, and points to the internal side of the measuring channel 1. This permits virtually complete filling of the measuring channel 1 with the test fluid. The venting of the measuring channel 1 takes place at least at one margin of the biosensor. The hydrophilic coating of the functional layer A3 likewise points to the interior of the channel and borders the entry orifice 2 of the measuring channel 1.

FIG. 1d shows by way of example a similar biosensor, except that in this case the venting notch 3 runs through the measuring channel 1 not transversely, as in the case of FIGS. 1a, 1b and 1c, but instead longitudinally. In this case the venting of the measuring channel 1 takes place towards the end of the biosensor.

This depiction should not be considered conclusive; instead, within the bounds of the invention, further designs are included.

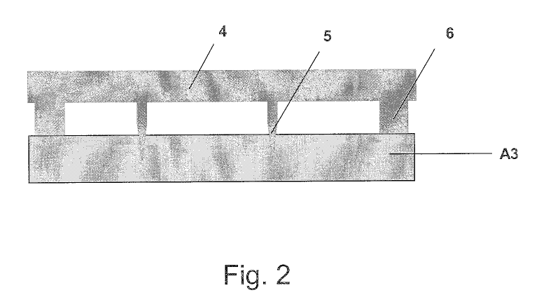

FIG. 2 shows diagrammatically one exemplary production of a venting notch. For that purpose the functional layer A3 is guided advantageously through a rotary punching tool with a punching cylinder 4, the punching cylinder being provided with a punching blade which represents a continuous circulating cutter 5. In production, this punching blade 5 partially penetrates the material of the functional layer A3, and displaces the material to form the venting notch 3. The depth of penetration of the cutter 5 into the functional layer A3 is determined by the difference in height between the spacers 6, known as the bearer rings, and the length of the cutters 5.

(Shown on the left adjacent to the right-hand punching blade 5 is a second blade which produces a further notch in the functional layer A3. This notch, after the punching of the functional layer A3, is found on another biosensor.)

### Test Methods

### Surface Tension and Contact Angle Measurement

The measurement of the contact angle with water and of the surface tension on solid surfaces takes place in accordance with EN 828:1997 using a G2/G402 instrument from Krüss GmbH. The surface tension is determined by the Owens-Wendt-Rabel&Kaeble method, by measuring the contact angle with deionized water and diiodomethane. The values are obtained in each case from the averaging of four results.

### Functional Test

To assess the transport characteristics of an aqueous test fluid, a capillary test is carried out. This is done by holding a test fluid consisting of deionized water and 1% by weight of naphthol red to the application orifice of the biosensor. The transport of the test fluid in the hydrophilic region is observed by means of a video camera. The channel test is also carried out after the biosensors under test have been stored at 23° C., 40° C. and 70° C., in order to test the ageing resistance and storage stability.

As the test fluid, use is likewise made of biological fluids such as blood. Biological fluids such as blood, however, are less suited as test fluid, since they are subject to fluctuations in properties. For example, the viscosity of blood fluctuates very sharply. The viscosity of blood is dependent on the haematocrit value.

### Thickness Measurement

The thickness, or the hydrophilic and hydrophobic coating applied, is measured by way of the optical method of reflectometry with a NanoCalc 2000 UV/Vis from Mikropack and a microscope from Leitz. This method utilizes the difference in the refractive indices of the different materials. The refractive index of the particular coating must be determined prior to measurement. For the PET base film a refractive index of 1.46 is used.

### Bond Strength

The peel strength (bond strength) was tested in a method based on PSTC-1. A strip of the pressure-sensitive adhesive tape 2 cm wide is adhered to the test substrate, such as a ground steel plate or a PET plate, for example, by applying the tape and running a 5 kg roller back and forth over it five times. The plate is clamped in and the self-adhesive strip is pulled by its free end in a tensile testing machine under a peel angle of 180° at a speed of 300 mm/min; the force required in order to pull the strip is measured. The results are reported in N/cm and are averaged over three measurements. All of the measurements were conducted at room temperature.

### Holding Power

The test was carried out in a method based on PSTC-7. A strip of the pressure-sensitive adhesive tape 1.3 cm wide is adhered to a polished steel plaque over a length of 2 cm, by applying the tape and running a 2 kg roller back and forth over it twice. The plaques are equilibrated for 30 minutes under test conditions (temperature and humidity), but without a load. Then the test weight is hung on, producing a shearing stress parallel to the bond surface, and a measurement is made of the time taken for the bond to fail. If a holding power time of 10 000 minutes is reached, the test is terminated before the bond fails.

### Microshear Travel

A strip of the pressure-sensitive adhesive tape 1 cm wide is adhered to a polished steel plaque (test substrate) over a length of 5 cm, by applying the strip to the plaque and rolling a 2 kg roller back and forth over it three times. Double-sided adhesive tapes are lined on the reverse with a 50 μm aluminium foil. The test strip is reinforced with a PET film 190 μm thick and then cut off flush using a fixing device. The edge of the reinforced test strip projects 1 mm beyond the edge of the steel plaque. The plaques are equilibrated for 15 minutes under test conditions (40° C., 50% relative humidity) in the measuring instrument, but without a load. Then the 500 g test weight is hung on, producing a shearing stress parallel to the bond surface. A micro-travel recorder records the shear travel in graph form as a function of time.

The microshear travel μS1 reported is the shear path after 15 minutes of weight loading. After the measuring time of 15 minutes under weight load, the weight is carefully removed from the sample and then the relaxation is observed for a further 15 minutes. After 15 minutes without a weight load (relaxation) the microshear travel μS2 is ascertained. From the two measurement values, the microshear travel ratio μS2/μS1 is formed. This ratio is a measure of the elasticity of the pressure-sensitive adhesive.

The intention of the text below is to illustrate the invention by means of a number of examples, without wishing thereby to restrict the invention unnecessarily.

### EXAMPLES

### Example 1

A biosensor is produced by lamination from the functional layers A1, A2 and A3.

The construction of the functional layer A1 is as follows: a 250 μm PET film, Hostaphan WO from Mitsubishi Polyesterfilm GmbH, is printed with the interconnects, using a conductive graphite paste E3455 from Ercon Inc. In the region of the measuring space, the reactive layer is then applied to the working electrode, said reactive layer consisting of the active component glucose dehydrogenase, the coenzyme NAD+, the mediator 1,10-phenanthroline, and a hydroxyethylcellulose binder. For the functional tests (fluid transport), the coating of the interconnects and of the reactive layer is omitted for the sake of simplicity. For the functional layer A3, the 100 μm Hostaphan® RN 100 PET film from Mitsubishi Polyesterfilm GmbH is corona-pretreated on one side and then coated over its full area, using a halftone roller, with a solution consisting of 0.5% by weight of Rewopol® SB DO 75 (sodium salt of diisooctylsulphosuccinic acid), from Evonik GmbH, in ethanol. The coating is dried in a drying tunnel at 120° C. After drying, the thickness of application is 25 nm.

For the functional layer A2, first of all the pressure-sensitive adhesive is prepared. For this purpose, a reactor conventional for free-radical polymerization is charged with 8 kg of acrylic acid, 45 kg of n-butyl acrylate, 3 kg of t-butyl acrylate and 60 kg of acetone. After nitrogen gas has been passed through the reactor for 45 minutes with stirring the reactor is heated to 58° C. and 20 g of azoisobutyronitrile (AIBN, Vazo 6®, DuPont) are added. Subsequently the external heating bath is heated to 75° C. and the reaction is carried out constantly at this external temperature. After a reaction time of 1 h a further 20 g of AIBN are added. After 3 h and 6 h the mixture is diluted with 10 kg of acetone/isopropanol (97:3) each time. In order to reduce the residual initiators, after 8 h and after 10 h, 100 g portions of bis(4-tert-butylcyclohexanyl) peroxydicarbonate (Perkadox 16®, Akzo Nobel) are added each time. After a reaction time of 22 h the reaction is terminated and cooled to room temperature. After the polymerization the polymer is diluted with isopropanol to a solids content of 25% and then blended with 0.3% by weight of polyisocyanate (Desmodur N 75, Bayer) with stirring. Subsequently the polymer solution is coated using a comma bar onto both sides of the 50 μm Hostaphan WO polyester carrier from Mitsubishi Polyesterfilms GmbH, which is pretreated by means of corona, with 12 g/m² in each case. The coating is dried in a drying tunnel at 120° C.

A rotary punch is used to produce diecuts from the resulting pressure-sensitive adhesive tape, the diecuts having a cut-out measuring channel with a width of 1.0 mm and a length of 5.0 mm, the channel being open at one side (corresponding to FIG. 1c). To produce the venting notch, the functional layer A3 furnished with the hydrophilic coating is likewise provided, using a rotary punch, with a continuous notch, in the course of which the punching blade penetrates the film to a depth of 70 μm. Both punching operations on the functional layers A2 and A3 run simultaneously in the same punching machine, so that the two layers can be laminated with a precise fit. Lamination takes place as shown in FIG. 1b, and so the venting notch is located on the inside of the measuring channel 0.5 mm before the closed end of the measuring channel. The positioning is checked by means of a camera system.

The lamination of the functional layer Al to the assembly comprising functional layer A2 and A3, for the production of test specimens, is carried out by hand on the small scale. For production operation, this lamination will likewise take place inline in the diecutting operation. The individual biosensors are cut from the roll material in a downstream operation.

The venting notch allows the air to escape perfectly from the measuring channel, and so a high transport rate can be achieved with an aqueous test fluid when the measuring channel is filled. The transport behaviour of the test fluid in the biosensor specimens does not change even after storage at 40° C. or 70° C. for 6 weeks.

### COUNTEREXAMPLES

### Counterexample 1

The functional layers A1, A2 and A3 are produced in the same way as in Example 1, but the functional layer A3 is not given a venting notch. The biosensor is not functional: the test fluid is unable to flow into the measuring channel.

### Counterexample 2

The functional layers A1 and A3 are produced in the same way as in Example 1.

For the production of the functional layer A2, the standard pressure-sensitive adhesive tape tesa 4972 (double-sided pressure-sensitive adhesive tape; thickness 50 μm; 12 μm PET carrier film, 2×20 g/m² coating with resin-modified acrylate adhesive) from tesa AG is used. The production of the diecuts and the lamination of the individual functional layers take place as described in Example 1.

Initially the biosensor is functional: the measuring channel can be quickly filled with the test fluid. However owing to the cold flow of the pressure-sensitive adhesive, the rate of filling of the measuring channel subsides very soon after its storage time of 1 week at 70° C. After storage times of 3 weeks at 70° C. and of 6 weeks at 40° C., the biosensors are no longer functional.

Overview of the properties of the examples and counterexamples

| | | | |
|---|---|---|---|
| | | Counter | Counter |
| Unit | Example 1 | example 1 | example 2 |

Functional layer A1

| | | | | |
|---|---|---|---|---|
| Material | | PET | PET | PET |
| Thickness | μm | 250 | 250 | 250 |

Functional layer A2

| | | | | |
|---|---|---|---|---|
| Thickness of | μm | 75 | 75 | 50 |
| adhesive tape | | | | |
| Thickness of | μm | 50 | 50 | 12 |
| carrier film | | | | |
| Type of adhesive | | straight | straight | acrylate, |
| | | acrylate | acrylate | modified |
| Adhesive | g/m² | 2 × 12 | 2 × 12 | 2 × 20 |
| coatweight | | | | |
| Bond strength for | N/cm | 2.5 | 2.5 | 6.5 |
| steel | | | | |
| Shear deformation | μm | 38 | 38 | 250 |

Functional layer A3

| | | | | |
|---|---|---|---|---|
| Material | | PET | PET | PET |
| Thickness | μm | 100 | 100 | 100 |
| Type of imprint | | surfactant | surfactant | surfactant |
| Contact angle | ° | 21 | 21 | 21 |
| Surface tension | mN/m | 67 | 67 | 67 |
| Function test | | fully functional | No fluid | fully |
| | | | transport | functional |
| Function test after | | fully functional | — | no fluid |
| 6 weeks at 70° C. | | | | transport |

1. A biosensor for analysing biological fluids, comprising
a first functional layer (A1) as a base layer of the biosensor,
a second functional layer (A3) in form of a polymer film having at least partially a hydrophilic coating,
a double-sided pressure-sensitive adhesive tape (A2) embedded between the first and the second functional layer,
wherein the double-sided pressure-sensitive adhesive tape (A2) includes a measuring channel extending partially into the double-sided pressure-sensitive adhesive tape,
wherein second functional layer (A3) includes at least one venting notch facing the measuring channel of the double-sided pressure-sensitive adhesive tape, the venting notch enables venting of the measuring channel.

2. The biosensor according to claim 1, wherein the first functional layer (A1), the second functional layer (A3) and the double-sided pressure-sensitive adhesive tape (A2) align at least on one side and
wherein the measuring channel includes an orifice at the alignment side, the orifice enables applying a biological fluid,
the at least one venting notch is positioned approximately near the termination of the measuring channel, opposite to the orifice.

3. The biosensor according to claim 1, wherein second functional layer (A3) is composed of one of polyester, polyethylene, polypropylene, polyvinyl chloride, polyacrylate, polycarbonate and/or of corresponding laminates or coextrudates.

4. The biosensor according to claim 1, wherein the venting notch is an indentation in the polymer film of the second functional layer (A3).

5. The biosensor according to claim 4, wherein the venting notch has a depth of 10 μm up to a maximum of 90% of the thickness of the polymer film.

6. The biosensor according to claim 4, wherein the thickness of the polymer film of the second functional layer (A3) is 12 to 350 μm and preferably 25 to 150 μm.

7. The biosensor according to claim 1, wherein the hydrophilic coating of the second functional layer (A3) has a surface tension of at least 60 mN/m and a contact angle with water of less than 30°.

8. The biosensor according to claim 1, wherein the hydrophilic coating of the second functional layer (A3) comprises at least one surfactant.

9. The biosensor according to claim 1, wherein the hydrophilic coating of the second functional layer (A3) is composed of polyvinyl alcohol as binder and of a surfactant and wherein the coating prior to drying has a viscosity of 50 to 500 mpa*s and more preferably of 80 to 200 mpa*s.

10. The biosensor according to claim 9, wherein the hydrophilic coating is applied over the full area or partial area by means of a printing process to the polymer film of the second functional layer (A3).

11. The biosensor according to claim 9, wherein the thickness of the hydrophilic coating of the second functional layer (A3) is not more than 3 μm.

12. The biosensor according to claim 1, wherein the venting notch in the second functional layer A3 is produced in die- cutting using a slitting or punching tool.

13. The biosensor according to claim 1, wherein the pressure-sensitive adhesive tape (A2) is composed of a pressure-sensitive adhesive whose holding power at 25° C. and 70° C. under a weight load of 1000 g is greater than 10 000 min and whose shear deformation after 15 min at 40° C. under a load of 500 g is less than 130 μm.

14. The biosensor according to claim 1, wherein the first functional layer (A1) is provided with interconnects and at least partially with an analytical detection reagent.

15. A method for using a biosensor according to claim 1 as a medical sensor or diagnostic strip for analyzing a biological fluid.

16. The biosensor according to claim 3, wherein second functional layer (A3) is composed of one of polyester, polyethylene, polypropylene, polyvinyl chloride, polyacrylate, polycarbonate and/or of corresponding laminates or coextrudates, which are monoaxially or biaxially oriented.

17. The biosensor according to claim 5, wherein the venting notch has a depth from 40 μm up to a maximum of 75% of the thickness of the polymer film.

18. The biosensor according to claim 8, wherein the at least one surfactant is an anionic surfactant.

18. The biosensor according to claim 8, wherein the at least one surfactant is a surfactant based on a sulphosuccinic ester salt.

19. The biosensor according to claim 10, wherein the printing process, is flexographic printing.

20. The biosensor according to claim 11, wherein the thickness of the hydrophilic coating of the second functional layer (A3) is not more than 1.5 μm.

21. The biosensor according to claim 13, wherein the shear deformation after 15 min at 40° C. under a load of 500 g is less than 80 μm.
